(19) United States
(12) Patent Application Publication
(10) Pub. No.: US 2025/0182283 A1
(43) Pub. Date: Jun. 5, 2025

(54) DYNAMIC IMAGE PROCESSING DEVICE, DYNAMIC IMAGE PROCESSING METHOD, AND RECORDING MEDIUM

(71) Applicant: Konica Minolta, Inc., Tokyo (JP)

(72) Inventor: Seiji NOMURA, Tokyo (JP)

(21) Appl. No.: 18/962,191

(22) Filed: Nov. 27, 2024

(30) Foreign Application Priority Data
Dec. 5, 2023 (JP) 2023-205184

(51) Int. Cl. G06T 7/00; G16H 30/40

(52) U.S. Cl. CPC G06T 7/0014; G16H 30/40; G06T 2207/10116; G06T 2207/30004

(57) ABSTRACT

A dynamic image processing device includes a hardware processor. The hardware processor acquires a dynamic image, judges a disease candidate based on the dynamic image, and determines a storage location of the dynamic image based on the disease candidate. In one embodiment, the hardware processor judges the disease candidate based on an analysis result of the dynamic image.

### CROSS-REFERENCE TO RELATED APPLICATIONS

The entire disclosure of Japanese Patent Application No. 2023-205184, filed on Dec. 5, 2023, is incorporated herein by reference in its entirety.

### BACKGROUND OF THE INVENTION

### Technical Field

The present invention relates to a dynamic image processing device, a dynamic image processing method, and a recording medium.

### Description of Related Art

Conventionally, a case collection (teaching file) is created for the purpose of improving the knowledge of students and medical residents.

In addition, a case collection is often created mainly using still images.

Currently, a dynamic image is also beginning to be used in case collections.

In addition, Japanese Unexamined Patent Publication No. 2023-27550 describes searching for a similar case image from a dynamic image.

However, since the amount of information of the dynamic image is larger than that of the still image, the dynamic image cannot be efficiently organized, and it takes time and effort to create the teaching file.

### SUMMARY OF THE INVENTION

An object of the present invention is to more easily construct teaching file information using a dynamic image.

To achieve at least one of the abovementioned objects, according to an aspect of the present invention, a dynamic image processing device reflecting one aspect of the present invention includes, a hardware processor,
wherein the hardware processor,
   acquires a dynamic image,
   judges a disease candidate based on the dynamic image, and
   determines a storage location of the dynamic image based on the disease candidate.

Further, according to another aspect of the present invention, a dynamic image processing method performed in a dynamic image processing device that processes a dynamic image, the method including:
acquiring a dynamic image,
judging a disease candidate based on the dynamic image, and
determining a storage location of the dynamic image based on the disease candidate.

Further, according to another aspect of the present invention, a non-transitory computer readable recording medium storing a program that causes a hardware processor of a computer of a dynamic image processing device that processes a dynamic image to perform:
acquiring a dynamic image,
judging a disease candidate based on the dynamic image, and
determining a storage location of the dynamic image based on the disease candidate.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating an overall configuration of a dynamic image processing system in an embodiment of the present invention; and

FIG. 2 is a flowchart illustrating disease candidate judgment processing.

### DETAILED DESCRIPTION

Hereinafter, one or more embodiments of the present invention will be described with reference to the drawings. However, the scope of the invention is not limited to the disclosed embodiments.

In the following, embodiments of the present invention will be described in detail with reference to the drawings. However, the scope of the invention is not limited to the illustrated examples.

### Configuration of Dynamic Image Processing System 100

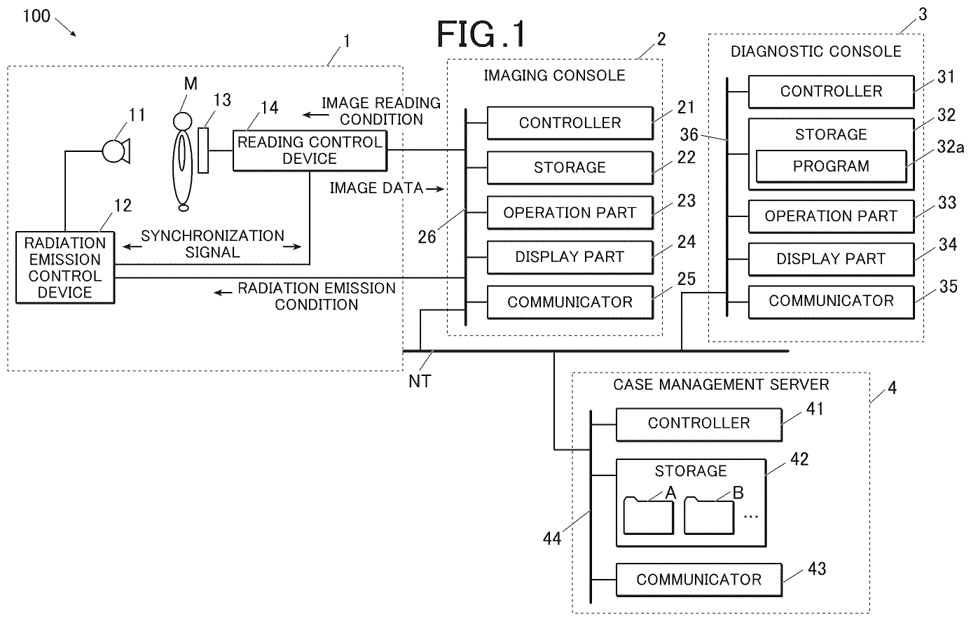

FIG. 1 illustrates an overall configuration of a dynamic image processing system 100 according to the present embodiment.

As shown in FIG. 1, the dynamic image processing system 100 is configured such that an imaging device 1 and an imaging console 2 are connected by a communication cable or the like, and the imaging console 2, a diagnostic console 3 as a dynamic image processing device, and a case management server 4 are connected via a communication network NT such as a local area network (LAN).

### Configuration of Imaging Device 1

The imaging device 1 is, for example, an imaging unit that images a dynamic state of a subject having periodicity, such as a change in form of expansion and contraction of lungs due to respiratory motion and pulsation of a heart.

Dynamic imaging is performed by repeatedly emitting pulsed radiation, such as X-rays, to a subject at intervals of a predetermined time (pulse emission) or continuously emitting radiation without a break to a subject at a low dose rate (continuous emission), thereby generating a plurality of images. That is, the dynamic imaging means that the dynamic state of a target portion having periodicity is continuously radiographed along the time axis.

Note that the dynamic imaging may be performed using not only radiation such as X-rays but also ultrasound waves or magnetism. The dynamic imaging includes moving image capturing, but does not include capturing of a still image while displaying a moving image.

A series of images obtained by dynamic imaging is referred to as a dynamic image.

The dynamic image can be acquired by imaging using a semiconductor image sensor such as a flat panel detector (FPD), for example.

Further, examples of a dynamic image includes a moving image but does not include still images captured while displaying a moving image.

Images constituting a dynamic image are called frame images. In the embodiment(s) described below, dynamic imaging of a chest is performed by pulse emission as an example. In the following embodiment(s), a case in which a subject M is a chest of a person who is the target of the examination is described as an example, but the present invention is not limited thereto.

A radiation source 11 is disposed at a position facing a radiation detector 13 with the subject M interposed therebetween. The radiation source 11 irradiates the subject M with radiation (X-rays) under the control of a radiation emission control device 12.

The radiation emission control device 12 is connected to the imaging console 2 and controls the radiation source 11 on the basis of radiation emission conditions input from the imaging console 2 to perform radiography.

The radiation emission conditions include, for example, a pulse rate, a pulse width, a pulse interval, the number of imaging frames per imaging, a value of an X-ray tube current, a value of an X-ray tube voltage, and a type of an additional filter.

The pulse rate is the number of times that radiation is emitted per second, and matches a frame rate described below. The pulse width is a period of time (duration) of one radiation emission. The pulse interval is a period of time from start of one radiation emission to start of the next radiation emission, and matches a frame interval described below.

The radiation detector 13 is constituted of a semiconductor image sensor such as an FPD.

The FPD includes, for example, a glass substrate, and a plurality of detection elements (pixels) are arranged in a matrix at predetermined positions on the substrate. The detection elements detect radiation emitted from the radiation source 11 and transmitted through at least the subject M in accordance with an intensity of the radiation, convert the detected radiation into electric signals, and accumulate the electric signals.

Each pixel includes a switching portion such as a thin film transistor (TFT), for example.

There are an indirect conversion FPD that converts X-rays into electric signals with photoelectric conversion element(s) via scintillator(s) and a direct conversion FPD that directly converts X-rays into electric signals. Either of these can be used.

According to the present embodiment, the pixel value (signal value) of the image data generated in the radiation detector 13 is a density value and is higher as the amount of transmitted radiation is greater.

The radiation detector 13 is arranged so as to face the radiation source 11 with the subject M in between.

A reading control device 14 is connected to the imaging console 2.

The reading control device 14 controls the switching portion of each pixel of the radiation detector 13 on the basis of the image reading condition input from the imaging console 2, switches the reading of the electric signal accumulated in each pixel, reads the electric signal accumulated in the radiation detector 13, and acquires image data. This image data is a frame image.

Then, the reading control device 14 assigns an identification ID and a frame number, and outputs the acquired frame image to the imaging console 2.

The image reading condition is, for example, a frame rate, a frame interval, a pixel size, an image size (matrix size), or the like.

The frame rate is the number of frame images acquired per second, and matches with the pulse rate. The frame interval is the time from the start of the operation of acquiring one frame image to the start of the operation of acquiring the next frame image, and matches with the pulse interval.

Here, the radiation emission control device 12 and the reading control device 14 are connected to each other and exchange synchronization signals with each other to synchronize the radiation emission operation and the image reading operation.

### Configuration of Imaging Console 2

The imaging console 2 outputs radiation emission conditions and image reading conditions to the imaging device 1 to control the radiography and the reading of the radiation image by the imaging device 1.

As illustrated in FIG. 1, the imaging console 2 includes a controller 21 (hardware processor), a storage 22, an operation part (operator) 23, a display part (display) 24, and a communicator 25, and these components are connected to each other by a bus 26.

The controller 21 includes a central processing unit (CPU), a random access memory (RAM), and the like. The CPU of the controller 21 reads a system program and various processing programs stored in the storage 22 according to the operation of the operation part 23, develops the programs in the RAM, executes various processes including an imaging control process according to the developed programs, and centrally controls the operation of each unit of the imaging console 2 and the radiation irradiation operation and the reading operation of the imaging device 1.

The storage 22 includes a nonvolatile semiconductor memory, a hard disk and the like. The storage 22 stores various programs executed by the controller 21, parameters necessary for execution of processing by the programs, or data such as processing results. For example, the storage 22 stores a program for executing imaging control processing. The various programs are stored in the form of readable program codes, and the controller 21 sequentially executes operations according to the program codes.

Specifically, the storage 22 stores a series of frame images (dynamic image) which are output from the imaging device 1 and to which identification IDs and frame numbers are assigned.

The storage 22 also stores imaging order information. The imaging order information is attached to a series of frame images (dynamic image) and stored in the storage 22.

The imaging order information includes radiation emission conditions (described above), image reading conditions (described above), information regarding a person as a target of the examination, examination information, and the like.

The information regarding the target of the examination includes, for example, the name, height, weight, age, and sex of the target.

The examination information is, for example, an imaging site (chest or the like), a diagnosis target (ventilation, pulmonary blood flow, or the like), an examination purpose (lung cancer, pneumonia, or the like), or the like.

The operation part 23 includes a keyboard including cursor keys, number input keys, and various function keys, and a pointing device such as a mouse, and outputs, to the controller 21, an instruction signal input by a key operation on the keyboard or a mouse operation. Furthermore, the operation part 23 may include a touch screen on the display screen of the display part 24, and in this case, outputs an instruction signal input via the touch screen to the controller 21.

The person who performs the imaging inputs the imaging order information by using the operation part 23.

The display part 24 is configured by a monitor such as a liquid crystal display (LCD) and a cathode ray tube (CRT), and displays an input instruction from the operation part 23, data, and the like according to an instruction of a display signal input from the controller 21.

The communicator 25 includes a LAN adapter, a modem, a terminal adapter (TA), and the like, and controls data transmission and reception to and from each device connected to the communication network NT.

### Configuration of Diagnostic Console 3

The diagnostic console 3 (dynamic image processing device) acquires the dynamic image from the imaging console 2 and displays the dynamic image. As described above, the dynamic image is accompanied by the imaging order information.

As shown in FIG. 1, the diagnostic console 3 is configured to include a controller 31 (hardware processor), a storage 32, an operation part 33, a display part 34, and a communicator 35, and each unit is connected by a bus 36.

The controller 31 includes a CPU, a RAM, and the like. In response to operation of the operation part 33, the CPU of the controller 31 reads a system program or various processing programs stored in the storage 32, loads the program in the RAM, and executes various processes in accordance with the loaded program. The CPU of the controller 31 reads a program 32a stored in the storage 32, loads the program 32a to the RAM, and executes image display processing (described below) according to the loaded program 32a.

In addition, the controller 31 functions as a dynamic image acquirer that acquires a dynamic image.

In addition, the controller 31 functions as a disease judgment section that judges a disease candidate based on the dynamic image.

In addition, the controller 31 functions as a storage location determination section that determines a storage location of the dynamic image based on the disease candidate.

The storage 32 includes a nonvolatile semiconductor memory, a hard disk and the like. The storage 32 stores a program 32a for executing image display processing in the controller 31, various programs, parameters necessary for execution of processing by the programs, processing results, and the like. These various programs are stored in the form of readable program codes, and the controller 31 sequentially executes operations in accordance with the program codes.

Furthermore, the storage 32 stores the dynamic image acquired from the imaging console 2 and accompanying imaging order information.

The operation part 33 includes a keyboard including cursor keys, number input keys, and various function keys, and a pointing device such as a mouse, and outputs, to the controller 31, an instruction signal input by a key operation on the keyboard or a mouse operation. Furthermore, the operation part 33 may include a touch screen on the display screen of the display part 34, and in this case, outputs an instruction signal input via the touch screen to the controller 31.

The display part 34 includes a monitor such as an LCD or a CRT, and performs various displays in accordance with an instruction of a display signal input from the controller 31.

The display part 34 functions as a display part for comparably displaying the first dynamic image and the second dynamic image that includes the interpolation image.

The communicator 35 includes a LAN adapter, a modem, a TA, and the like, and controls data transmission and reception to and from each device connected to the communication network NT.

The communicator 35 functions as a transmitter that transmits a dynamic image to the outside.

### Configuration of Case Management Server 4

The case management server 4 is an apparatus that stores and manages the dynamic images (cases) transmitted from the diagnostic console 3. As described above, the dynamic image is accompanied by the imaging order information.

As illustrated in FIG. 1, the case management server 4 includes a controller 41 (hardware processor), a storage 42, and a communicator 43, and each unit is connected by a bus 44.

The controller 41 includes a CPU, a RAM, and the like. The CPU of the controller 41 reads a system program and various processing programs stored in the storage 42, loads the programs in the RAM, and executes various processes in accordance with the loaded programs.

The storage 42 includes a nonvolatile semiconductor memory, a hard disk, and the like. The storage 42 stores various programs, parameters required for execution of processing by the programs, and data such as processing results. These various programs are stored in the form of readable program codes, and the controller 41 sequentially executes operations in accordance with the program codes.

Further, in the storage 42, the dynamic image transmitted from the diagnostic console 3 is stored in the storage location (storage location 42A, storage location 42B, . . . ) determined by the controller 31. For example, a dynamic image of “disease candidate; lung cancer” is stored in the storage location 42A, and a dynamic image of “disease candidate; pneumonia” is stored in the storage location 42B.

The communicator 43 includes a LAN adapter, a modem, a TA, and the like, and controls data transmission and reception to and from each device connected to the communication network NT.

### Disease Candidate Judgment Processing

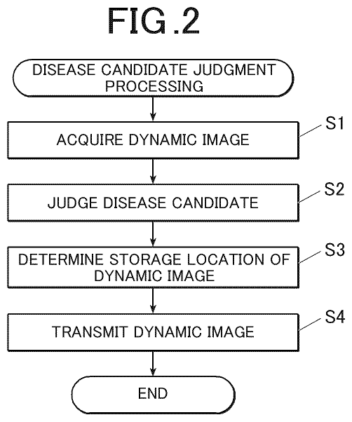

Next, disease candidate judgment processing in the diagnostic console 3 will be described with reference to FIG. 2.

The disease candidate judgment processing is processing for judging a disease candidate from the dynamic image.

It is assumed that the dynamic image has already been stored in the storage 32 of the diagnostic console 3.

First, the controller 31 acquires the dynamic image from the storage 32 (step S1).

As described above, the dynamic image is accompanied by the imaging order information.

Note that if the imaging order information is not used in step S2 or later, the controller 31 might not acquire the imaging order information.

Next, the controller 31 judges a disorder candidate from the dynamic image (step S2).

For example, the controller 31 extracts feature amounts from the dynamic image by using machine learning and judges disease names having a degree of similarity of a predetermined value or more to be disease candidates.

Furthermore, for example, the controller 31 may use machine learning to extract the imaging site, diagnostic target, and examination purpose included in the imaging order information in addition to the feature amounts from the dynamic images and judge the disease candidate.

Furthermore, for example, the controller 31 may refer to the examination purpose included in the imaging order information and add the disease information included in the examination purpose to the disease candidate.

Note that a learning model used for machine learning is stored in the storage 32 before the disease candidate judgment processing.

Furthermore, although the disease candidate is judged using machine learning in the above description, it is not limited thereto.

Next, the controller 31 determines the storage location of the dynamic image based on the disease candidate (step S3). After determining the storage location, the controller 31 transmits the dynamic image to the storage location via the communicator 35 (step S4).

For example, the controller 31 determines the dynamic image of the same disease candidate to be stored in the same storage location. In a case where the storage location corresponding to the disease candidate is created in advance, such storage location is used as the storage location. When the storage location corresponding to the disease candidate has not been created, the storage location is newly created in the storage section 42, and such storage location is used as the storage location.

### Others

Although the disease candidate judgment processing is executed by the controller 31 of the diagnostic console 3 in the above description, it may be executed by the controller 41 of the case management server 4.

The number of diagnostic consoles 3 and the number of case management servers 4 are not limited to one, and may be two or more. In addition, the diagnostic console 3 and the case management server 4 are not limited to being disposed inside the hospital, and may be disposed outside the hospital.

### Effect

As described above, the dynamic image processing device (diagnostic console 3) includes the dynamic image acquirer (controller 31) that acquires dynamic images, the disease judgment section (controller 31) that judges disease candidates on the basis of the dynamic image, and the storage location determination section (controller 31) that determines the storage location of the dynamic image on the basis of the disease candidate.

Therefore, the teaching file information can be constructed more easily.

In recent years, significant advances have been made in machine learning including AI. Therefore, it is considered that disease candidates judged from dynamic images will increase. Therefore, it is assumed that a lot of time and effort will be necessary for a user to manually determine the storage location and create the teaching file for each disease candidate that will increase. The diagnostic console 3 can prevent such a situation.

The disease judgment section (controller 31) judges the disease candidate on the basis of an analysis result of the dynamic image.

Therefore, the teaching file information can be constructed more easily.

In addition, the disease judgment section (controller 31) judges the disease candidate based on the imaging order information.

Therefore, the teaching file information can be constructed more easily.

That is, if the examination for the specific disease is performed, it cannot necessarily be said that the patient suffers from the specific disease. For example, in a case where the examination for “lung cancer” of a certain patient is performed, there is a possibility that the patient does not have “lung cancer”. On the other hand, “pneumonia” may be judged as the disease candidate for the patient from the dynamic image. In such a case, “lung cancer” that is the examination purpose can also be added as the disease candidate.

The disease judgment section (controller 31) judges the disease candidate by using machine learning.

Therefore, the teaching file information can be constructed more easily.

Further, the dynamic image processing device (diagnostic console 3) includes the transmitter (communicator 35) that transmits the dynamic image to the outside, and the storage location determination section determines a transmission destination to which the dynamic image is transmitted based on the disease candidate.

Therefore, even when a plurality of diagnostic consoles 3 are connected to the dynamic image processing system 100, the teaching file information can be more easily constructed.

The dynamic image processing method includes, in a dynamic image processing device that processes dynamic images, a dynamic image acquiring step (step S1) that acquires dynamic images, a disease judgment step (step S2) that judges the disease candidate on the basis of the dynamic image, and a storage location determination step (step S3) that determines the storage location of the dynamic image on the basis of the disease candidate.

Therefore, the teaching file information can be constructed more easily.

Furthermore, the program causes the computer of the dynamic image processing device (diagnostic console 3) that processes the dynamic image to function as the dynamic image acquirer (controller 31) that acquires the dynamic image, the disease judgment section (the controller 31) that judges the disease candidate based on the dynamic image, and the storage location determination section (controller 31) that determines the storage location of the dynamic image based on the disease candidate.

Therefore, the teaching file information can be constructed more easily.

Note that the description according to the present embodiment is an example of a suitable dynamic image processing system according to the present invention, and the present invention is not limited to this.

For example, although the example of the construction of the teaching file information has been disclosed in the above description, it is also possible to determine an external network folder as a storage location of the dynamic image, store the dynamic image in the folder, and request a specialist physician to perform image reading such as remote image reading.

Although a hard disk, a semiconductor nonvolatile memory, or the like is used in the above description as a computer-readable medium storing the program according to the present invention, the present invention is not limited to this example. Other applicable computer-readable media include portable recording media such as CD-ROM. In addition, a carrier wave is also applied as a medium for providing data of the program according to the present invention via a communication line.

In addition, the detailed configuration and the detailed operation of each device constituting the dynamic image processing system 100 can be appropriately changed without departing from the scope of the present invention.

Although embodiments of the present invention have been described and shown in detail, the disclosed embodiments are made for purposes of illustration and example only and not limitation. The scope of the present invention should be interpreted by terms of the appended claims.

The entire disclosure of Japanese Patent Application No. 2023-205184, filed on Dec. 5, 2023, including description, claims, drawings and abstract is incorporated herein by reference.

What is claimed is:

1. A dynamic image processing device comprising:
a hardware processor,
wherein the hardware processor,
acquires a dynamic image,
judges a disease candidate based on the dynamic image, and
determines a storage location of the dynamic image based on the disease candidate.

2. The dynamic image processing device according to claim 1, wherein the hardware processor judges the disease candidate based on an analysis result of the dynamic image.

3. The dynamic image processing device according to claim 2, wherein the hardware processor judges the disease candidate based on imaging order information.

4. The dynamic image processing device according to claim 1, wherein the hardware processor judges the disease candidate using machine learning.

5. The dynamic image processing device according to claim 1, wherein the hardware processor determines that the dynamic image is stored in the storage location storing the dynamic image of the same disease candidate.

6. The dynamic image processing device according to claim 1, further comprising a transmitter that transmits the dynamic image to the outside,
wherein the hardware processor determines a transmission destination to which the dynamic image is to be transmitted based on the disease candidate.

7. A dynamic image processing method, performed in a dynamic image processing device that processes a
dynamic image, the method comprising:
acquiring a dynamic image,
judging a disease candidate based on the dynamic image, and determining a storage location of the dynamic image based on the disease candidate.

8. A non-transitory computer readable recording medium storing a program that causes a hardware processor of a computer of a dynamic image processing device that processes a dynamic image to perform:
acquiring a dynamic image,
judging a disease candidate based on the dynamic image, and
determining a storage location of the dynamic image based on the disease candidate.
